(19) United States
(12) Patent Application Publication
(10) Pub. No.: US 2026/0174835 A1
(43) Pub. Date: Jun. 25, 2026

(54) ARTICLE OF MANUFACTURE FOR SEMAGLUTIDE COMPOSITIONS

(71) Applicant: ORBICULAR PHARMACEUTICAL TECHNOLOGIES PRIVATE LIMITED, Hyderabad (IN)

(72) Inventors: Patel Bhaveshkumar VALLABHBHAI, Hyderabad (IN); Raghu KANNEKANTI, Hyderabad (IN)

(21) Appl. No.: 19/357,370

(22) Filed: Oct. 14, 2025

(30) Foreign Application Priority Data
Oct. 14, 2024 (IN) 202441077621

(51) Int. Cl. A61K 38/26; A61K 9/00; A61K 9/08; A61K 47/02; A61K 47/10

(52) U.S. Cl. CPC A61K 38/26; A61K 9/0019; A61K 9/08; A61K 47/02; A61K 47/10

(57) ABSTRACT

The objective invention provides a parenteral composition of semaglutide or a pharmaceutically acceptable salt thereof for subcutaneous administration, having a strength and composition such that it can be administered multiple times from single article of manufacture, for use in treating type 2 diabetes mellitus and for weight loss, thereby providing benefit to the patient.

The present invention provides a parenteral composition of semaglutide or a pharmaceutically acceptable salt thereof for subcutaneous administration, having a strength and composition such that it can be administered multiple times from single article of manufacture, for use in treating type 2 diabetes mellitus and for weight loss, thereby providing benefit to the patient.

### BACKGROUND OF THE INVENTION

Semaglutide (Formula I) is a human GLP-1 receptor agonist (or GLP-1 analog). The main protraction mechanism of semaglutide is albumin binding, facilitated by modification of position 26 lysine with a hydrophilic spacer and a C18 fatty di-acid. Semaglutide is also modified in position 8 to provide stabilization against degradation by the enzyme dipeptidyl-peptidase 4 (DPP-4). A minor modification at position 34 helps ensure the attachment of only one fatty di-acid. The molecular weight of semaglutide is 4113.58 g/mol.

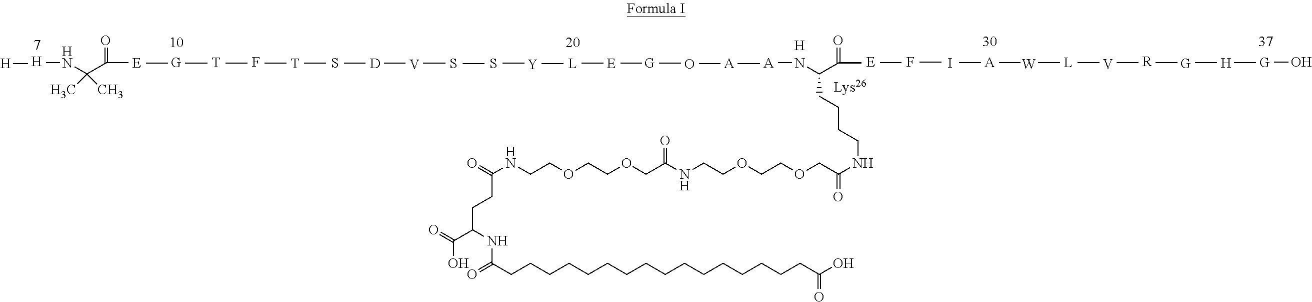

Semaglutide is a GLP-1 analogue with 94% sequence homology to human GLP-1. Semaglutide acts as a GLP-1 receptor agonist that selectively binds to and activates the GLP-1 receptor, the target for native GLP-1. GLP-1 is a physiological hormone that has multiple actions on glucose, mediated by the GLP-1 receptors. The principal mechanism of protraction resulting in the long half-life of semaglutide is albumin binding, which results in decreased renal clearance and protection from metabolic degradation. Furthermore, semaglutide is stabilized against degradation by the DPP-4 enzyme, as discussed above. Semaglutide reduces blood glucose through a mechanism where it stimulates insulin secretion and lowers glucagon secretion, both in a glucose-dependent manner. Thus, when blood glucose is high, insulin secretion is stimulated, and glucagon secretion is inhibited. The mechanism of blood glucose lowering also involves a minor delay in gastric emptying in the early postprandial phase. Semaglutide lowers fasting and postprandial blood glucose and reduces body weight. Both first- and second-phase insulin secretion are increased in patients with type 2 diabetes treated with semaglutide. It lowers high blood glucose concentrations by stimulating insulin secretion and lowering glucagon secretion in a glucose-dependent manner. With semaglutide, the insulin secretion rate in patients with type 2 diabetes was similar to that of healthy subjects.

Injectable semaglutide has been approved as Wegovy® by the USFDA and is indicated in combination with a reduced calorie diet and increased physical activity to reduce the risk of major adverse cardiovascular events (cardiovascular death, non-fatal myocardial infarction, or non-fatal stroke) in adults with established cardiovascular disease and either obesity or overweight. It is also approved to reduce excess body weight and maintain weight reduction long term in adults and pediatric patients aged 12 years and older with obesity, and in adults with overweight in the presence of at least one weight-related comorbid condition. It is also approved for the treatment of noncirrhotic metabolic dysfunction-associated steatohepatitis (MASH), formerly known as nonalcoholic steatohepatitis (NASH), with moderate to advanced liver fibrosis (consistent with stages F2 to F3 fibrosis) in adults. It has also been approved by the USFDA as Ozempic®, which is indicated for use as an adjunct to diet and exercise to improve glycemic control in adults with type 2 diabetes mellitus. It is also approved to reduce the risk of major adverse cardiovascular events (cardiovascular death, non-fatal myocardial infarction or non-fatal stroke) in adults with type 2 diabetes mellitus and established cardiovascular disease. It is also approved to reduce the risk of sustained eGFR decline, end-stage kidney disease, and cardiovascular death in adults with type 2 diabetes mellitus and chronic kidney disease.

The currently approved Wegovy® product requires weekly administration of a dose ranging from 0.25 mg/week to 2.4 mg/week. Wegovy® is available as single-dose pen in strengths of 0.25 mg/0.5 mL, 0.5 mg/0.5 mL, 1 mg/0.5 mL, 1.7 mg/0.75 mL, and 2.4 mg/0.75 mL. The availability of single dose pens makes it cumbersome to procure and stock medication for longer periods of time, such as a 3 or 6-months' supply or more, and such as when the patient may be travelling.

The Ozempic® product is also administered once weekly, with approved doses ranging from 0.25 mg/week to 2 mg/week. Ozempic® is provided as 2 mg/3 mL (0.68 mg/ml), 4 mg/3 mL (1.34 mg/mL) and 8 mg/3 mL (2.68 mg/mL) prefilled disposable, single-patient-use pens, such that 4 doses of 0.25 mg and 2 doses of 0.5 mg, or 4 doses of 0.5 mg are available per pen. Thus, the currently commercially available products can store only a maximum of 4 doses of semaglutide.

It should also be noted that administration of a single dose of Wegovy® requires administration of 0.5 to 0.75 ml of the solution, while that for Ozempic® requires administration of about 0.2 ml to 0.75 ml. Large subcutaneous injection volumes are associated with pain. In this sense, the maximum volume generally accepted is around 1.5 ml, although volumes of up to 3 ml are well tolerated when injected in the abdomen. If lower volume could be used to administer a similar dose of semaglutide, it would provide a benefit to the patient.

U.S. Pat. No. 8,536,122 discloses semaglutide and its uses in heretofore approved uses. U.S. Pat. No. 11,318,191 discloses liquid pharmaceutical compositions comprising 0.01 to 100 mg/mL of semaglutide, 0.5-10 mg/ml of semaglutide, no more than 0.1% (w/w) phenol and 7-9.5 mg/ml sodium chloride, but U.S. Pat. No. 11,318,191 does not exemplify or demonstrate stable embodiments of semaglutide compositions at or above a concentration of 25 mg/mL. Similarly, IN539320 discloses semaglutide composition containing semaglutide, in a concentration of 0.1-10 mg/ml; phosphate, in a concentration of more than 15 mM and less than or equal to 45 mM; and a pH of 7-8. IN257402 discloses semaglutide compositions of strength 6.25 mg/ml.

U.S. Pat. No. 10,335,462B2 discloses to an improved use of GLP-1 agonists in therapy which showed improved reduction of HbA1c and reduction of body weight. Further discloses, the GLP-1 agonist is administered in an amount which provides an improved a) reduction in HbA1c or b) reduction in body weight compared to administration of 1.8 mg liraglutide or less, such as 0.8 mg liraglutide or less, per day. However, the art is silent on a Multi dose pen device which can be administered for more than a month.

WO2023/238017 discloses a liquid pharmaceutical composition comprising 4.8 mg/mL semaglutide, 0.35% w/w phenol, 1.42 mg disodium phosphate dihydrate, 14 mg propylene glycol and water. It also discloses a cartridge comprising a liquid pharmaceutical composition of semaglutide wherein semaglutide is in a concentration of 3 mg/ml to 25 mg/ml and the phenol is in concentration of 0.11% w/w to 0.5% w/w with buffer, pharmaceutically acceptable excipients and water. It also describes a multi-dose delivery device consisting a cartridge suitable to deliver multiple variable doses of liquid pharmaceutical composition of semaglutide, such that the device delivers the dose of semaglutide in the range of 0.25 mg to 2 mg for the treatment of diabetes, and a dose in the range of 0.25 mg to 2.4 mg for the treatment of obesity. This publication provides injectable compositions of semaglutide in multiple and variable doses in a pen injector. Specifically, it discloses only semaglutide of strength up to 25 mg/ml, and exemplifies pen injectors with a maximum of 15 doses/cartridge. The publication provides only 1 week stability data for a composition of strength 4.8 mg/ml, and it is not understood, nor is any indication provided in the publication, if such a composition would be stable over a longer period, such that the product remains stable until the 15 exemplified doses in the cartridge are used. The one-week stability data also does not confirm that a solution containing high concentration of semaglutide and high concentration of phenol can remain stable over a long period of time, such as for 24 months or more.

WO2024148155A1 discloses a composition, methods and implantable devices that improve patient compliance by releasing a desired therapeutic agent such as semaglutide for a long time period, such as 1 year or more. The semaglutide concentration (w/w) is more than 1%, such as about 1.1% w/w to about 35% w/w. However, the invention is silent on Multi dose pen device which can be administered for more than a month.

WO2020127476A1 and WO2021123228A1 discloses pharmaceutical compositions containing a Glucagon like peptide-1 (GLP-1) analogue. In particular, the present invention provides pharmaceutical compositions containing one or more GLP-1 analogues such as liraglutide and semaglutide, optionally in a combination with one or more other active substances. The concentration of GLP-1 may be from 0.1 to 100 mg/mL. The composition discloses Liraglutide and Semaglutide compositions and exemplifies a Liraglutide composition with maleic acid and sorbitol. The publication further concludes that sorbitol-containing excipient solution exerts a stabilizing effect on the liraglutide structure and slows down the tendency for fibre formation. However, the art is silent on devices such as a Multi dose pen device which can be administered for more than a month.

CN114469877 discloses a semaglutide implant and the preparation method thereof, which can effectively prolong the action time of the semaglutide in vivo, reduce the injection times of the medicament and the bioavailability of the medicament, improve the adaptability of patients and facilitate the clinical use and the acceptance of the patients. However, the art is silent on devices such as Multi dose pen device which can be administered for more than a month.

The present invention provides a stable solution of semaglutide or a pharmaceutically acceptable salt thereof at a concentration of less than 100 mg/mL and more than 27.5 mg/mL that is suitable for subcutaneous injection, wherein the concentration of semaglutide or a pharmaceutically acceptable salt thereof allows the use of a smaller volume of the solution to be administered. As a result, it becomes a convenient means for including a larger number of doses in prefilled syringes and cartridges that are used in pens, thereby making it convenient for the patient to stock doses, without the need to replace or buy additional doses of the medication every week or every 2 weeks i.e., more cost-effective treatment. Particularly, the semaglutide or a pharmaceutically acceptable salt thereof at a concentration of less than 100 mg/mL and more than 27.5 mg/mL of the present invention are suitable for filling in large number of doses, i.e. multi-doses, in a PFS and/or cartridge, such that doses for up to 1 month, 2 months, 3 months, 4 months or more can be provided and stored without the concern of degradation. This increases patient compliance, at the very least. It also ensures that the patient does not run out of doses and continues to have uninterrupted supply of daily or weekly doses for his/her use. For chronic conditions such as type 2 diabetes, as well as for use in treatment of obesity, it is important not to miss out on doses, for optimal therapeutic efficacy of semaglutide or a pharmaceutically acceptable salt thereof. The present invention thus provides a suitable means for such uninterrupted use through multi-dose pens containing semaglutide or a pharmaceutically acceptable salt thereof at a concentration of less than 100 mg/mL and more than 27.5 mg/mL.

### OBJECT OF THE INVENTION

An object of the present invention relates to an article of manufacture comprising a container comprising a composition of semaglutide or a pharmaceutically acceptable salt thereof at a concentration of less than 100 mg/mL and more than 27.5 mg/mL.

An object of the present invention relates to an article of manufacture according to the above embodiment, wherein the container comprises semaglutide or a pharmaceutically acceptable salt thereof composition which comprises semaglutide at one or more doses of 0.25 mg, 0.5 mg, 1 mg, 1.7 mg, 2 mg and 2.4 mg.

Another object of the present invention relates to the article of manufacture of the above embodiments, wherein the container is selected from a bottle, a vial, a syringe, a pen, a pump, a multidose needle syringe, a multidose pen, a jet injector, a pre-filled syringe, or combinations thereof.

Another object of the present invention relates to the article of manufacture of the above embodiments, wherein the container is a multidose pen.

Another object of the present invention relates to a multi-dose pen comprising multiple doses of semaglutide or a pharmaceutically acceptable salt thereof composition which comprises semaglutide at one or more doses of 0.25 mg, 0.5 mg, 1 mg, 1.7 mg, 2 mg and 2.4 mg.

Another object of the present invention to provide a multi-dose pen comprising a composition of semaglutide or a pharmaceutically acceptable salt thereof used for the treatment of semaglutide sensitive diseases.

### SUMMARY OF THE INVENTION

The present invention relates to an article of manufacture comprising a container comprising semaglutide or a pharmaceutically acceptable salt thereof at a concentration of less than 100 mg/mL and more than 27.5 mg/mL.

The present invention relates to an article of manufacture according to the above embodiment, container comprises semaglutide or a pharmaceutically acceptable salt thereof composition.

The present invention relates to an article of manufacture according to the above embodiments, wherein the container comprises semaglutide or a pharmaceutically acceptable salt thereof composition which comprises semaglutide one or more doses of 0.25 mg, 0.5 mg, 1 mg, 1.7 mg, 2 mg and 2.4 mg.

The present invention relates to an article of manufacture of the above embodiments, wherein the container is selected from a bottle, a vial, a syringe, a pen, a pump, a multidose needle syringe, a multidose pen, a jet injector, a pre-filled syringe, or combinations thereof.

The present invention relates to an article of manufacture of the above embodiments, wherein the container is a multidose pen.

The present invention relates to the multidose pen according to the above embodiments, wherein each dose comprises of about 750 μl or less.

The present invention relates to the multidose pen according to the above embodiments for use in any one or more purpose like initiation, escalation and maintenance treatment.

The present invention relates to the multidose pen according to the above embodiments comprising one or more semaglutide doses of 0.25 mg, 0.5 mg, 1 mg, 1.7 mg, 2 mg and 2.4 mg.

The present invention relates the multidose pen according to the above embodiments comprising one or more semaglutide doses of 0.25 mg, 0.5 mg, 1 mg, 1.7 mg, 2 mg and 2.4 mg for one, two, three and up to 09 months.

### DETAILED DESCRIPTION OF INVENTION

The article of manufacture according to any of the embodiments herein, wherein the composition is used for the treatment of semaglutide or a pharmaceutically acceptable salt thereof sensitive diseases or disorders. The article of manufacture includes a container and a composition comprising a semaglutide or a pharmaceutically acceptable salt thereof at a concentration of less than 100 mg/mL and more than 27.5 mg/mL and other composition details. Proteins and peptides, like semaglutide or a pharmaceutically acceptable salt thereof, are inherently unstable molecules. Due to their reliance on tertiary structure for activity, proteins and peptides are often sensitive to a range of external stressors including temperature, pH change, light and agitation, which can lead to irreversible degradation or aggregation.

A Semaglutide or a pharmaceutically acceptable salt thereof solution provided by the present invention has a strength of less than 100 mg/mL and more than 27.5 mg/mL. Typically, the invention is a stable semaglutide or a pharmaceutically acceptable salt thereof solution of semaglutide or a pharmaceutically acceptable salt thereof at a concentration of less than 100 mg/mL and more than 27.5 mg/mL that allows lower volumes of the solution to be injected.

The “stable pharmaceutical composition” or “stable composition” described herein include active pharmaceutical ingredient with one or more pharmaceutical acceptable excipient(s) having physical and chemical stability.

The “stable composition of semaglutide or a pharmaceutically acceptable salt thereof” described herein comprises one or more pharmaceutical acceptable excipient(s) having physical and chemical stability.

The term “stability” or “stable” as used herein includes both physical and chemical stability. Stability parameters include but are not limited to potency, stable pH value and other physico-chemical parameters. Peptide compositions are widely used as medicine. Most of the peptide drugs are formulated as liquid injectable compositions. The pharmaceutical composition of such peptides is required to be stable or to have a shelf life of several years in order to be suitable for use as medicine. However, peptide compositions are inherently unstable due to sensitivity towards physical and chemical degradation. To ensure the product's safety and efficacy, the peptide composition must meet defined quality and stability during and/or immediately after manufacture as well at the end of their designated shelf lives or during storage.

The term “initiation dose” or “initiation” as used herein refers to the first administration of a drug, often involving a loading dose designed to quickly achieve a therapeutic level in the patient.

The term “escalation dose” or “escalation” as used herein refers to the process of gradually increasing the drug dose after initiation to reach an effective or optimal therapeutic level while monitoring safety and efficacy.

The term “maintenance dose” or “maintenance” as used herein refers to the ongoing administration of a drug at a stable dose after escalation, intended to maintain a therapeutic effect without increasing toxicity. Maintenance doses are typically lower or optimized doses that sustain the desired drug concentration over time.

The term “therapeutically effective dose” refers to any dose that produces a desired effect in a subject, such as alleviating symptoms, slowing disease progression, or preventing disease onset. This dose may be administered multiple times over a period of time to achieve the desired effect.

The excipients used in the compositions of the present invention may include one or more of buffer, pH adjusting agent, stabilizer, isotonicity agent, preservative and a carrier. In preferred embodiments, the compositions are aqueous in nature, i.e. the carrier is water, used in the form of sterile water for injection.

One or more buffers that may be used in the compositions of the present invention may be selected from disodium hydrogen phosphate dihydrate, acetate, carbonate, citrate, phosphate, or slats thereof, glycine, lysine, arginine, glycylglycine, tris(hydroxymethyl)-aminomethan, tris or combinations or mixtures thereof. The buffer is used in an amount so as to maintain pH of the semaglutide or a pharmaceutically acceptable salt thereof solution at the pH of optimum solubility and stability of semaglutide or a pharmaceutically acceptable salt thereof. Buffer(s) are added to the compositions to optimize solubility and stability of semaglutide or a pharmaceutically acceptable salt thereof by adjusting the pH. However, the strength of the buffer is kept as low as possible to avoid pain upon injection. In one embodiment of the present invention, the concentration of phosphate buffer is limited to about 10 mM. In another embodiment, the concentration of citrate buffer is kept lower than about 7.3 mM to avoid an increased sensation of pain. In another embodiment, buffering agent is disodium hydrogen phosphate dihydrate.

The semaglutide or a pharmaceutically acceptable salt thereof at a concentration of less than 100 mg/mL and more than 27.5 mg/mL solutions of the present invention may include pH adjusting agent(s) selected from sodium hydroxide and hydrochloric acid of suitable molarity, to adjust the pH of the solution to a pH that provides optimum solubility and stability of semaglutide or a pharmaceutically acceptable salt thereof. Typically, such a pH is around the isoelectric point (pI) of the peptide or protein that is to be included in a solution. The isoelectric point of semaglutide is 5.4, and it has low solubility in the pH range of pH 2-6. Also, use of acidic pH would cause adverse reactions at the site of injection. Therefore, the pH is ideally such that it is closer to the pI but also towards neutral pH so as to remain tolerable upon subcutaneous injection. Thus, the semaglutide or a pharmaceutically acceptable salt thereof at a concentration of less than 100 mg/mL and more than 27.5 mg/mL solutions of the present invention have a pH in the range of about 6.5 to about 7.8. Preferably, the pH may be around 7.4.

The semaglutide or a pharmaceutically acceptable salt thereof at a concentration of less than 100 mg/mL and more than 27.5 mg/mL solutions of the present invention may include one or more stabilizers selected from amino acids such as histidine, methionine, cysteine, arginine, glycine, proline, lysine, glutamic acid, isoleucine, phenylalanine, tyrosine, and aspartic acid.

The stabilizers may also include pharmaceutically acceptable excipients such as povidone, polyethylene glycol, propylene glycol, polyethylene glycol 300/400, glycerin, ethanol and mixtures thereof. These polymers work as solubilizing scaffolds that counter hydrophobicity, and help in keeping the peptide in solution. In addition to increasing the efficacy of semaglutide or a pharmaceutically acceptable salt thereof, stabilizers help in maintaining the product at a desired viscosity. In another embodiment, stabilizer is propylene glycol.

The semaglutide or a pharmaceutically acceptable salt thereof at a concentration of less than 100 mg/mL and more than 27.5 mg/mL solutions of the present invention may include saccharides and sugar alcohols as stabilizers. Examples include but are not limited to trehalose, maltose, sucrose, dextran and the like, and mixtures thereof.

Chelating agents like EDTA and its salts; surfactants such as polysorbates, Cremophor EL, N-methyl-2-pyrrolidone (NMP); antioxidants such as ascorbic acid, butylayed hydroxy anisole (BHA), butylated hydroxy toluene (BHT), Vitamin E TPGS, and the like and mixtures thereof, may also be used in the semaglutide or a pharmaceutically acceptable salt thereof at a concentration of less than 100 mg/mL and more than 27.5 mg/mL solutions of the present invention.

The semaglutide or a pharmaceutically acceptable salt thereof solutions of the present invention which are for subcutaneous injection are required to be isotonic so as to prevent injection site reactions, such as haemolysis, redness of skin, irritation and tissue damage. Typically, the isotonicity of the solutions of the present invention is in the range of about 280 mOsm to about 320 mOsm. The solutions of the present invention may include one or more of sodium chloride, mannitol, dextrose, glycerol, propylene glycol and mixtures thereof as isotonicity agents. In another embodiment, tonicity agent is propylene glycol.

Preservatives are required to inhibit the growth of microorganisms in multi-dose injectable compositions such as those of the present invention. They ensure long term stability and efficacious use of the semaglutide injections.

As used herein, an “article of manufacture” is a product that is made and, in some cases, that can be sold. In some embodiments, the term can refer to compositions contained in articles of packaging, such as in a container.

Also provided herein is an article of manufacture comprising a compound of the disclosure or a pharmaceutically acceptable salt, tautomer, stereoisomer, mixture of stereoisomers, prodrug, or deuterated analog thereof in a suitable drug delivery device. Drug delivery device can be pen injector, disposable pen injector, reusable pen injector and the like.

In another embodiment, the invention provides an article of manufacture comprising the pharmaceutical composition as described herein.

In one embodiment, the pharmaceutical composition in the article of manufacture comprises multiple doses of a semaglutide pharmaceutical composition according to the present invention wherein such pharmaceutical composition is a single dose form that comprises semaglutide in one of the dosage ranges specified herein.

In another embodiment, articles of manufacture mean the semaglutide can be packaged as articles of manufacture containing packaging material with instruction as in a kit, and a semaglutide composition provided herein, which is used for treatment, prevention or amelioration of one or more symptoms associated with semaglutide sensitive diseases or disorders.

In another embodiment, an article of manufacture comprises the pharmaceutical composition comprising a GLP-1 receptor agonist described herein for the treatment, prevention and/or diagnosis of the disorders described herein. The article of manufacture comprises a container and a label or package insert on or associated with the container. The containers may be formed from a variety of materials such as glass or plastic. The container holds the pharmaceutical composition comprising the semaglutide which is by itself or when combined with another composition effective for treating, preventing and/or diagnosing the condition.

The article of manufacture is a drug delivery device or system. The article of manufacture can be disposable or reusable.

Disposable means the article of manufacture can be used more than once for a single patient only with the same primary container closure or drug container or cartridge in place and is discarded after the drug container is empty.

Reusable means article of manufacture can be used more than once for a specified single patient only with replaceable primary container closure or drug container or cartridge.

In one embodiment, the article of manufacture comprises one or more parts as housing, barrel, body, cap, cartridge or container, cartridge holder, injection button, dose knob, dose selector, dose window, dose display, dose pointer, last dose nut, last dose identifier, needle, number sleeve, spring, piston, piston guide, piston holder, and the like.

One embodiment of the present invention relates to an article of manufacture of the above embodiments, wherein the container is selected from a bottle, a vial, a syringe, a pen, a pump, a multidose needle syringe, a multidose pen, a jet injector, a pre-filled syringe, or combinations thereof.

The present invention relates to an article of manufacture of the above embodiments, wherein the article of manufacture is a multidose pen.

In one embodiment, the article of manufacture optionally comprises a different mechanism during dialing and dosing of pharmaceutical composition specified herein. In another embodiment, the article of manufacture comprises one or more mechanism as a locking mechanism, dial mechanism, feedback mechanism and the like.

In one embodiment, the article of manufacture optionally comprises a locking mechanism. In another embodiment, the article of manufacture comprises a locking mechanism to provision for an extra dose. In another embodiment, the article of manufacture comprises a different mechanism for delivering the specified number of doses.

In one embodiment, the article of manufacture comprises a dial mechanism which meters each dose. The dial mechanism can be dialing the article of manufacture at a proximal end of the device, wherein the one or more part of article of manufacture takes place to complete the dial mechanism further during dosing also for the dosing mechanism the one or more part takes place.

In another embodiment, the article of manufacture comprises the feedback mechanism such as audible, tactile or visual at setting the dose, at dosing, and when dosing is complete.

In one embodiment, the article for manufacture comprises a dose counter that can be present at proximal end or distal end or middle of article of manufacture. In another embodiment the article of manufacture displays the number of doses present in the article of manufacture. In another embodiment the article of manufacture displays the number of doses used by patient.

In one embodiment, the article for manufacture for subcutaneous injection comprises needle in the range of 30 to 34 gauge and 3.5 mm to 5 mm in length.

In one embodiment the present invention comprises semaglutide, and one or more preservative with pharmaceutically acceptable excipient(s).

Another embodiment of the present invention comprises semaglutide, and phenol with one or more pharmaceutically acceptable excipient(s).

Another embodiment of the present invention comprises semaglutide, and cresol with one or more pharmaceutically acceptable excipient(s).

Semaglutide concentration means a pharmaceutical composition or formulation used herein comprising semaglutide at a concentration of less than 100 mg/mL and more than 27.5 mg/mL.

In one embodiment, the present invention comprises semaglutide or a pharmaceutically acceptable salt thereof in a concentration of more than 27.5 mg/mL to less than 60 mg/mL, one or more preservative with pharmaceutically acceptable excipient(s).

In one embodiment, the present invention comprises semaglutide or a pharmaceutically acceptable salt thereof in a concentration of about 30 mg/mL, and one or more preservative with pharmaceutically acceptable excipient(s).

In one embodiment, the present invention is an article of manufacture comprising a container comprising a pharmaceutical composition for subcutaneous injection, comprising: semaglutide or a pharmaceutically acceptable salt thereof at a concentration more than 27.5 mg/mL and less than 100 mg/mL, one or more pharmaceutically acceptable excipient(s) including a buffer and an isotonicity agent, wherein said composition: is suitable for use in a multi-dose article of manufacture capable of delivering multiple doses of semaglutide for more than 4 doses from a single container.

In one embodiment, the present invention is an article of manufacture comprising a container comprising a pharmaceutical composition for subcutaneous injection, comprising: semaglutide or a pharmaceutically acceptable salt thereof at a concentration more than 27.5 mg/mL and less than 100 mg/mL, one or more pharmaceutically acceptable excipient(s) including a buffer and an isotonicity agent, wherein said composition: is suitable for use in a multi-dose article of manufacture capable of delivering multiple doses of semaglutide for at more than 8 doses from a single container.

In one embodiment, the present invention is an article of manufacture comprising a container comprising a pharmaceutical composition for subcutaneous injection, comprising: semaglutide or a pharmaceutically acceptable salt thereof at a concentration more than 27.5 mg/mL and less than 100 mg/mL, one or more pharmaceutically acceptable excipient(s) including a buffer and an isotonicity agent, wherein said composition: is suitable for use in a multi-dose article of manufacture capable of delivering multiple doses of semaglutide for at more than 12 doses from a single container.

In one embodiment, the present invention is an article of manufacture comprising a container comprising a pharmaceutical composition for subcutaneous injection, comprising: semaglutide or a pharmaceutically acceptable salt thereof at a concentration more than 27.5 mg/mL and less than 100 mg/mL, one or more pharmaceutically acceptable excipient(s) including a buffer and an isotonicity agent, wherein said composition: is suitable for use in a multi-dose article of manufacture capable of delivering multiple doses of semaglutide for at least 24 doses from a single container.

In another embodiment, the present invention comprises a method of administering semaglutide for treating semaglutide sensitive diseases or disorders in a patient in need thereof, the method comprising: providing the article of manufacture as specified in the above embodiments, wherein the device comprises a concentration more than 27.5 mg/mL and less than 100 mg/mL semaglutide, and subcutaneously administering to the patient, a therapeutically effective dose of semaglutide from the article of manufacture at intervals of about once per week for more than 1 month.

In another embodiment, the present invention comprises a method of administering semaglutide for treating semaglutide sensitive diseases or disorders in a patient in need thereof, the method comprising: providing the article of manufacture as specified in the above embodiments, wherein the device comprises a concentration more than 27.5 mg/mL and less than 100 mg/mL semaglutide, and subcutaneously administering to the patient, a therapeutically effective dose of semaglutide from the article of manufacture at intervals of about once per week for more than 2 months.

In another embodiment, the present invention comprises a method of administering semaglutide for treating semaglutide sensitive diseases or disorders in a patient in need thereof, the method comprising: providing the article of manufacture as specified in the above embodiments, wherein the device comprises a concentration more than 27.5 mg/mL and less than 100 mg/mL semaglutide, and subcutaneously administering to the patient, a therapeutically effective dose of semaglutide from the article of manufacture at intervals of about once per week for more than 3 months.

In another embodiment, the present invention comprises a method of administering semaglutide for treating semaglutide sensitive diseases or disorders in a patient in need thereof, the method comprising: providing the article of manufacture as specified in the above embodiments, wherein the device comprises a concentration more than 27.5 mg/mL and less than 100 mg/mL semaglutide, and subcutaneously administering to the patient, a therapeutically effective dose of semaglutide from the article of manufacture at intervals of about once per week for at least 6 months.

In another embodiment, the present invention is an article of manufacture, comprising: a composition of
a) semaglutide or a pharmaceutically acceptable salt thereof;
b) propylene glycol;
c) phosphate buffer;
d) preservative; and
e) one or more pharmaceutically acceptable excipient(s);

wherein the semaglutide or a pharmaceutically acceptable salt thereof at a concentration of less than 100 mg/mL and more than 27.5 mg/ml;

wherein the composition is stable for at least 6 months at 2-8° C. and 25° C./60% RH;

and wherein the article of manufacture is multi-dose pen.

In another embodiment, the present invention comprises semaglutide or a pharmaceutically acceptable salt thereof at a concentration of less than 100 mg/mL and more than 27.5 mg/mL, and one or more preservative with pharmaceutically acceptable excipient(s).

In another embodiment, the present invention comprises an article of manufacture comprising a container comprising semaglutide or a pharmaceutically acceptable salt thereof at a concentration of less than 100 mg/mL and more than 27.5 mg/mL, and one or more preservative selected from phenol, meta cresol and the like, with pharmaceutically acceptable excipient(s).

In another embodiment, the present invention comprises a multi-dose container comprising semaglutide or a pharmaceutically acceptable salt thereof at a concentration of less than 100 mg/mL and more than 27.5 mg/mL, one or more preservative selected from group comprises of phenol, meta cresol and the like with pharmaceutically acceptable excipient(s).

In another embodiment, the present invention comprises a multi-dose container comprising semaglutide or a pharmaceutically acceptable salt thereof at a concentration of less than 100 mg/mL and more than 27.5 mg/mL, and one or more preservative selected from phenol, meta cresol and the like, with pharmaceutically acceptable excipient(s), wherein the container provide multiple doses from single container.

In one embodiment, the article of manufacture comprises more than 27.5 mg/mL to less than 100 mg/mL semaglutide, wherein the article of manufacture comprises a container or cartridge for delivering same or different multiple doses of a semaglutide composition.

The semaglutide or a pharmaceutically acceptable salt thereof at a concentration of less than 100 mg/mL and more than 27.5 mg/mL compositions of the present invention are packaged in prefilled syringes and cartridges that can be used in pens, which are helpful in providing multiple doses of semaglutide or a pharmaceutically acceptable salt thereof over a period of at least 3 months and more. Essentially, the multidose pens containing the semaglutide or a pharmaceutically acceptable salt thereof at a concentration of less than 100 mg/mL and more than 27.5 mg/mL compositions of the present invention are capable of holding at least 16 doses that can provide once weekly dosing of semaglutide or a pharmaceutically acceptable salt thereof for at least 4 months. In other embodiments, the multidose pens of the present invention contain 20 to 36 doses, i.e., once weekly doses for 5 to 9 months, and the like.

In one embodiment, the multidose pen according to the above embodiments is used for any one or more purpose like initiation, escalation and maintenance treatment.

In one embodiment, the multidose pen according to the above embodiments is used for initiation treatment.

In one embodiment, the multidose pen according to the above embodiments is used escalation treatment.

In one embodiment, the multidose pen according to the above embodiments is used maintenance treatment.

In one embodiment, the multidose pen according to the above embodiments comprises one or more semaglutide doses of 0.25 mg, 0.5 mg, 1 mg, 1.7 mg, 2 mg and 2.4 mg.

In one embodiment, the multidose pen according to the above embodiments comprises one or more semaglutide doses of 0.25 mg, 0.5 mg, 1 mg, 1.7 mg, 2 mg and 2.4 mg for one, two, three and up to 09 months.

In another embodiment the multidose pen of a semaglutide composition according to the above embodiments comprises for initiation 0.25 mg of 4 doses and 8 doses of 0.5 mg for maintenance, and the like.

In another embodiment the multidose pen of a semaglutide composition according to the above embodiments comprises for initiation 0.25 mg of 4 doses and 12 doses of 0.5 mg for maintenance, and the like.

In another embodiment the multidose pen of a semaglutide composition according to the above embodiments comprises for initiation 0.25 mg of 4 doses and 16 doses of 0.5 mg for maintenance, and the like.

In another embodiment the multidose pen of a semaglutide composition according to the above embodiments comprises doses for initiation, escalation, and maintenance, and the like.

In another embodiment the multidose pen of a semaglutide composition according to the above embodiments comprises doses for initiation 0.25 mg of 4 doses, 4 doses of 0.5 mg for escalation and 4 doses of 1 mg for maintenance, and the like.

In another embodiment the multidose pen of a semaglutide composition according to the above embodiments comprises for initiation 0.25 mg of 4 doses, 4 doses of 0.5 mg for escalation, 4 doses of 1 mg for escalation and 4 doses of 2 mg for maintenance, and the like.

In another embodiment the multidose pen of a semaglutide composition according to the above embodiments comprises for initiation 0.25 mg of 4 doses, 4 doses of 0.5 mg for escalation, 4 doses of 1 mg for escalation and 4 doses of 1.7 mg for maintenance, and the like.

In another embodiment the multidose pen of a semaglutide composition according to the above embodiments comprises for initiation 0.25 mg of 4 doses, 4 doses of 0.5 mg for escalation, 4 doses of 1 mg for escalation and 8 doses of 1.7 mg for maintenance, and the like.

In another embodiment the multidose pen of a semaglutide composition according to the above embodiments comprises for initiation 0.25 mg of 4 doses; 4 doses of 0.5 mg for escalation; 4 doses of 1 mg for escalation and 4 doses of 1.7 mg for escalation and 4 doses of 2.4 mg for maintenance, and the like.

In another embodiment the multidose pen of a semaglutide composition according to the above embodiments comprises for initiation 0.25 mg of 4 doses; 4 doses of 0.5 mg for escalation; 4 doses of 1 mg for escalation and 4 doses of 1.7 mg for escalation and 8 doses of 2.4 mg for maintenance, and the like.

In another embodiment the multidose pen of a semaglutide composition according to the above embodiments for maintenance treatment.

In another embodiment the multidose pen of a semaglutide composition according to the above embodiments comprises 12 doses for 3 months, and the like.

In another embodiment the multidose pen of a semaglutide composition according to the above embodiments comprises 24 doses for 6 months, and the like.

In another embodiment the multidose pen of a semaglutide composition according to the above embodiments comprises 0.5 mg of 12 doses for 3 months, and the like.

In another embodiment the multidose pen of a semaglutide composition according to the above embodiments comprises 0.5 mg of 24 doses for 6 months, and the like.

In another embodiment the multidose pen of a semaglutide composition according to the above embodiments comprises 1 mg of 12 doses for 3 months, and the like.

In another embodiment the multidose pen of a semaglutide composition according to the above embodiments comprises 1 mg of 24 doses for 6 months, and the like.

In another embodiment the multidose pen of a semaglutide composition according to the above embodiments comprises 2 mg of 12 doses for 3 months, and the like.

In another embodiment the multidose pen of a semaglutide composition according to the above embodiments comprises 2 mg of 24 doses for 6 months, and the like.

In another embodiment the multidose pen of a semaglutide composition according to the above embodiments comprises 1.7 mg of 12 doses for 3 months, and the like.

In another embodiment the multidose pen of a semaglutide composition according to the above embodiments comprises 1.7 mg of 24 doses for 6 months, and the like.

In another embodiment the multidose pen of a semaglutide composition according to the above embodiments comprises 2.4 mg of 12 doses for 3 months, and the like.

In another embodiment the multidose pen of a semaglutide composition according to the above embodiments comprises 2.4 mg of 24 doses for 6 months, and the like.

An article of manufacture includes a container and a composition comprising a semaglutide or a pharmaceutically acceptable salt thereof at a concentration of less than 100 mg/mL and more than 27.5 mg/mL and other composition details.

In one embodiment the present invention relates to the multidose pen according to embodiments, wherein the container or cartridge size is 0.5 mL to 20 mL.

In one embodiment the present invention relates to the multidose pen according to embodiments, wherein the container or cartridge size is 1 mL to 5 mL.

In one embodiment the present invention relates to the multidose pen according to embodiments, wherein each dose comprises about 750 μl or less.

In another embodiment the present invention relates to the multidose pen according to embodiments, wherein each dose comprises about 750 μl or less, 725 μl or less, 700 μl or less, 675 μl or less, 650 μl or less, 625 μl or less, 600 μl or less, 575 μl or less, 550 μl or less, 525 μl or less, 500 μl or less, 475 μl or less, 450 μl or less, 425 μl or less, 400 μl or less, 375 μl or less, 350 μl or less, 325 μl or less, 300 μl or less, 275 μl or less, 250 μl or less, 225 μl or less, 200 μl or less, 175 μl or less, 150 μl or less, 125 μl or less, 100 μl or less, 95 μl or less, 90 μl or less, 85 μl or less, 80 μl or less, 75 μl or less, 70 μl or less, 65 μl or less, 60 μl or less, 55 μl or less, 50 μl or less, 45 μl or less, 40 μl or less, 35 μl or less, 30 μl or less, 25 μl or less, 20 μl or less, 15 μl or less, 10 μl or less, 5 μl or less and the like.

In one embodiment the present invention relates to the multidose pen according to embodiments, comprising the design of multi-dose pen for delivery of doses from container as shown in Table 1:

TABLE 1
Design of multi-dose pen

| Composition | Dose | μl delivered | Total |
|---|---|---|---|
| concentration (mg/ml) | about (mg) | per dose | Dose counts |
| less than 100 mg/mL and | 0.25 mg | 750 μl | Less than |
| more than 27.5 mg/mL | to 2.4 mg | or less | 36 doses |

In one embodiment the present composition comprises osmolality less than about 320 mOsmol/kg.

In one embodiment the present composition comprises osmolality between about 220 to about 320 mOsmol/kg. In another embodiment the present composition comprises osmolality between about 260 to about 320 mOsmol/kg.

The pens that may be used to store and administer the semaglutide or a pharmaceutically acceptable salt thereof at a concentration of less than 100 mg/mL and more than 27.5 mg/mL compositions of the present invention are devices that are capable of storing multiple doses of the composition and are capable of subcutaneous administration. It includes devices that have the ability to adjust the dose that can be delivered, through means such as dials that can regulate the volume of solution that can be administered. Devices with digital display and dose counters may be used as well.

Compatibility of injectable solution with components and parts of the device which holds the solution is another critical aspect. Potential discoloring, deformation, and leachables from the packaging material due to incompatibility must be controlled critically. Leaching of alkaline materials from glass may cause an increase in solution pH. The reaction can be significant for unbuffered compositions, as the solubility and stability of semaglutide or a pharmaceutically acceptable salt thereof are sensitive to the pH change. Container-closure integrity before manufacturing and during storage is important to ensure and maintain integrity of the semaglutide or a pharmaceutically acceptable salt thereof solutions. The excipients and parameters described herein above help address these issues and thereby provide a stable, semaglutide or a pharmaceutically acceptable salt thereof at a concentration of less than 100 mg/mL and more than 27.5 mg/mL composition in multidose pens.

The United States Pharmacopoeia (USP) defines stability as “the ability of a product to retain its characteristics that it possessed during its manufacturing (physical, chemical, microbiological, therapeutic properties) within specified limits throughout its period of storage and use”. According to the ICH guidelines, pharmaceutical stability testing is defined as “systematic experiments conducted on pharmaceutical products to understand and provide evidence how the quality of a drug product varies under the influence of variety of environmental factors such as temperature, humidity and light, and to set re-test period for the drug, or a shelf life for the drug product and recommend good storage conditions”. In view of these, degradation products in a drug product are required to be evaluated and reported to the regulatory agencies. Further, USFDA requires parenteral compositions to be stable for a minimum period of 12 months (i.e. 48 weeks) at recommended storage condition, for approving the same for commercial use. For products to be commercially marketed in the US, there is an expectation that the products will comply to the standard throughout the shelf life.

The semaglutide or a pharmaceutically acceptable salt thereof at a concentration of less than 100 mg/mL and more than 27.5 mg/mL compositions of the present invention were found to be stable over their shelf life, when stored in PFS and cartridges at 2-8° C. The term “shelf life” refers to the amount of time the pharmaceutical composition may be stored without loss of potency and/or performance profile. In some embodiments of the present invention, shelf life refers to the amount of time the pharmaceutical composition may be stored without loss of about 4%, about 3%, about 2% or about 1% of the potency and/or performance of the active ingredient, i.e. semaglutide, or a pharmaceutically acceptable salt thereof when stored at refrigerated conditions, i.e. about 2° C. to about 8° C. The stable compositions provided herein are designed to have shelf life of at least 24 months.

In another embodiment the article of manufacture store at prior to first use, stored in a refrigerator between 36° F. to 46° F. (2° C. to 8° C.) and after first use of the article of manufacture, the article of manufacture store up to shelf-life at controlled room temperature (59° F. to 86° F.; 15° C. to 30° C.) or in a refrigerator (36° F. to 46° F.; 2° C. to 8° C.).

In another embodiment: the article of manufacture recommended storage is as shown in Table:

TABLE 2
Recommended Storage Conditions for the article of manufacture:

| | |
|---|---|
| Prior to first use | After first use |

| | | |
|---|---|---|
| Refrigerated | Room Temperature | Refrigerated |
| 36° F. to 46° F. | 59° F. to 86° F. | 36° F. to 46° F. |
| (2° C. to 8° C.) | (15° C. to 30° C.) | (2° C. to 8° C.) |

| | |
|---|---|
| Until expiration date | 84 days |

In another embodiment, the present invention is a multi-dose pen of semaglutide or pharmaceutically acceptable form composition sterilized by the methods known in the art and filled in suitable containers. In another embodiment container sterilization is done by the methods known in the art.

In another embodiment, the present invention is a multi-dose pen of semaglutide or pharmaceutically acceptable form composition; wherein the composition is administered by self-administer injection device.

In another embodiment, the present invention is a multi-dose pen of semaglutide or pharmaceutically acceptable form composition used for the treatment of semaglutide sensitive diseases or disorders selected from type 2 diabetes mellitus or obesity or to reduce the risk of major adverse cardiovascular events or noncirrhotic metabolic dysfunction-associated steatohepatitis (MASH) or chronic kidney disease in a patient in need thereof.

The present invention is further illustrated by reference to the following experiments which is for illustrative purpose only and does not limit the scope of the invention in any way.

### Example

TABLE 3
Composition Details

| | | Composi- | Composi- | Composi- |
|---|---|---|---|---|
| | Name of | tion 1 Qty. | tion 2 Qty. | tion 3 Qty. |
| Sr. | Ingredient | in % w/v | in % w/v | in % w/v |
| 1. | Semaglutide | 3.0 | 3.0 | 3.0 |
| 2. | Disodium Hydrogen | 0.142 | 0.142 | 0.142 |
| | Phosphate Dihydrate | | | |
| 3. | Propylene Glycol | 0.14 | 0.14 | 0.14 |
| 4. | Phenol | 0.55 | — | — |
| 5. | Benzyl Alcohol | — | 0.95 | — |
| 6. | Meta cresol | — | — | 0.3 |
| 7. | Sodium Hydroxide | Qs to pH | Qs to pH | Qs to pH |
| 8. | Hydrochloric Acid | Qs to pH | Qs to pH | Qs to pH |
| 9. | Water for Injection | Qs | Qs | Qs |

Qs—Quantity sufficient
Qty.—Quantity

### Manufacturing Procedure:

1) Required quantity of intended batch size of water for injection was transferred to the mixing vessel.
2) The nitrogen was purged till the dissolved oxygen (DO) content was less than 1 ppm.
3) Under constant stirring, dispensed quantities of disodium hydrogen phosphate dihydrate, propylene glycol and phenol/benzyl alcohol/meta cresol were added and stirred until the excipients were completely dissolved at specified rpm.
4) Under constant stirring, the dispensed quantity of semaglutide was added until the semaglutide had completely dissolved at specified rpm.
5) The pH was checked and adjusted if required, and the volume was made up to 100% with the remaining quantity of water for injection. The mixture was stirred for the specified time with specified rpm, and the final pH was checked and recorded. The temperature was then brought down to 2-8° C. (36-46° F.).
6) The solution was filtered with a sterilizing grade filter and stored at 2-8° C. (36-46° F.).
7) The filtered solution was filled into the cartridges. Nitrogen was purged pre-filling into the cartridges.

TABLE 4
Summarized stability results for the tested compositions:

| | | | | | | Composition 2 | | | | | | | |
|---|---|---|---|---|---|---|---|---|---|---|---|---|---|
| | Composition 1 | | | | | | 2- | 25° C./ | Composition 3 | | | | |
| Composition | | 2-8° C. | | 25° C./60% RH | | | 8° C. | 60% | | 2-8° C. | | 25° C./60% RH | |
| Condition | Initial | 1M | 6M | 1M | 6M | Initial | 1M | RH 1M | Initial | 1M | 6M | 1M | 6M |
| Description | CCS* | CCS* | CCS* | CCS* | CCS* | CCS* | CCS* | CCS* | CCS* | CCS* | CCS* | CCS* | CCS* |
| pH | 7.41 | 7.42 | 7.41 | 7.41 | 7.45 | 7.42 | 7.42 | 7.43 | 7.43 | 7.4 | 7.47 | 7.43 | 7.45 |
| Osmolality | 274 | 272 | 272 | 272 | 276 | 266 | 269 | 264 | 265 | 266 | 262 | 268 | 264 |
| Assay of | 100.2 | 97.9 | 98.2 | 96.2 | 92.4 | 98.2 | 91.4 | 88.2 | 103.3 | 98.9 | 99.7 | 100.2 | 94.8 |
| Semaglutide | | | | | | | | | | | | | |
| Aggregate | 0.03 | 0.06 | 0.04 | 0.12 | 0.18 | 0.04 | 0.06 | 0.12 | 0.03 | 0.06 | 0.03 | 0.17 | 0.2 |
| content | | | | | | | | | | | | | |
| Total | 0.18 | 0.32 | 0.26 | 0.69 | 3.21 | 1.04 | 7.06 | 10.95 | 0.24 | 0.48 | 0.39 | 0.86 | 3.46 |
| Impurities | | | | | | | | | | | | | |

CCS*- Clear colorless solution

TABLE 5
The design of multi-dose pen for delivery
of doses from container as below:

| Composition | Dose | μl delivered | Total |
|---|---|---|---|
| concentration (Mg/ml) | about (mg) | per dose | Dose counts |
| less than 100 mg/mL and | 0.25 mg | 750 μl | Less than |
| more than 27.5 mg/mL | to 2.4 mg | or less | 36 doses |

Surprisingly, present inventors found that the composition's 1 & 3 comprising semaglutide or a pharmaceutically acceptable salt thereof at a concentration of less than 100 mg/mL and more than 27.5 mg/mL with phenol (composition 1) and meta cresol (composition 3) have superior stability over benzyl alcohol (composition 2); and the maintained stability which was a surprising and unexpected development with the said semaglutide concentration. Further after 6 months at 2-8° C. & 25° C./60% RH, both physical and chemical stability is maintained.

TABLE 6
In-Use Stability Data for Composition 1

| | | Inuse_5° C._{—} | Inuse_30° C._{—} | Inuse_5° C._{—} | Inuse_30° C._{—} | Inuse_5° C._{—} | Inuse_30° C._{—} |
|---|---|---|---|---|---|---|---|
| Condition | Initial | 28 days | 28 days | 56 days | 56 days | 84 days | 84 days |
| Description | NA | Complies | Complies | Complies | Complies | Complies | Complies |
| pH | 7.42 | 7.4 | 7.41 | 7.4 | 7.43 | 7.41 | 7.42 |
| Assay of | 99.9 | 101 | 102.4 | 98.2 | 94.1 | 97.6 | 91.5 |
| semaglutide | | | | | | | |
| Assay of phenol | 100.2 | 99.8 | 100.1 | 100.2 | 99.9 | 100 | 99.8 |
| Total aggregate | 0.007 | ND | 0.03 | ND | 0.09 | ND | 0.19 |
| content | | | | | | | |

Related Substances

| | | | | | | | |
|---|---|---|---|---|---|---|---|
| Total | 0.04% | 0.18 | 1.15 | 0.2 | 2.17 | 0.23% | 3.67 |
| Impurities | | | | | | | |

TABLE 7
In-Use Stability Data for Composition 3

| | | Inuse_5° C._{—} | Inuse_30° C._{—} | Inuse_5° C_{—} | Inuse_30° C._{—} | Inuse_5° C._{—} | Inuse_30° C._{—} |
|---|---|---|---|---|---|---|---|
| Condition | Initial | 28 days | 28 days | 56 days | 56 days | 84 days | 84 days |
| Description | NA | Complies | Complies | Complies | Complies | Complies | Complies |
| pH | 7.43 | 7.41 | 7.43 | 7.42 | 7.43 | 7.41 | 7.43 |
| Assay of | 102.6 | 100.3 | 101.7 | 97.2 | 92.6 | 98.2 | 91.5 |
| semaglutide | | | | | | | |
| Assay of Meta | 98.8 | 99.1 | 99 | 99.7 | 99.8 | 99.3 | 98.9 |
| cresol | | | | | | | |
| Total | 0.007 | ND | 0.04 | ND | 0.1 | ND | 0.19 |
| aggregate | | | | | | | |
| content | | | | | | | |

Related Substances

| | | | | | | | |
|---|---|---|---|---|---|---|---|
| Total | 0.06% | 0.65 | 1.52 | 0.63 | 2.61 | 0.63 | 3.67 |
| Impurities | | | | | | | |

The in-use stability results for Semaglutide composition (with phenol and meta cresol as preservative) demonstrate that the formulation remains physically and chemically stable under both refrigerated (5° C.) and accelerated (30° C.) conditions for up to 84 days.

Overall, the results confirm that the semaglutide composition is stable for extended in-use periods under refrigerated conditions, maintaining specification compliance for up to three months (84 days).

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention.

We claim:

1. An article of manufacture comprising a container comprising pharmaceutical composition for subcutaneous injection, comprising: semaglutide or a pharmaceutically acceptable salt thereof at a concentration more than 27.5 mg/mL and less than 100 mg/mL, and one or more pharmaceutically acceptable excipient(s) including buffer and isotonicity agents, wherein said composition: is suitable for use in a multi-dose article of manufacture capable of delivering multiple doses of semaglutide from a single container.

2. The article of manufacture according to claim 1, wherein the semaglutide concentration is from more than 27.5 mg/mL to less than 60 mg/mL.

3. The article of manufacture according to claim 1, wherein the semaglutide concentration is about 30 mg/mL.

4. The article of manufacture according to claim 1, wherein the buffer is disodium hydrogen phosphate dihydrate and the isotonicity agent is propylene glycol.

5. The article of manufacture according to claim 1, further comprising preservative and one or more pharmaceutically acceptable excipient(s).

6. The article of manufacture according to claim 1, further comprising one or more preservative selected from phenol and meta cresol.

7. The article of manufacture according to claim 1, wherein the article of manufacture is a multi-dose pen.

8. The article of manufacture according to claim 1, wherein each dose comprises about 750 μl or less.

9. The article of manufacture according to claim 1, wherein the article of manufacture comprises semaglutide doses sufficient to dispense treatment consisting of initiation, escalation and maintenance doses.

10. The article of manufacture according to claim 1, comprising one or more semaglutide doses of 0.25 mg, 0.5 mg, 1 mg, 1.7 mg, 2 mg and 2.4 mg.

11. The article of manufacture according to claim 1, wherein the article of manufacture comprises semaglutide doses sufficient to dispense treatment for one, two, three and up to 9 months.

12. The article of manufacture according to claim 1, wherein the article of manufacture comprises semaglutide doses sufficient to dispense treatment for three to six months.

13. The article of manufacture according to claim 1, wherein the article of manufacture comprises semaglutide doses sufficient to dispense treatment multiple maintenance doses.

14. The article of manufacture according to claim 1, wherein the article of manufacture comprises less than 36 doses.

15. The article of manufacture according to claim 1, wherein the article of manufacture comprises less than 24 doses.

16. The article of manufacture according to claim 1, wherein the article of manufacture comprises less than 12 doses.

17. The article of manufacture according to claim 1, wherein the pharmaceutical composition has pH 7.0 to 7.8.

18. The article of manufacture according to claim 1, wherein the pharmaceutical composition has osmolality 220 to 320 mOsmol/kg.

19. The article of manufacture according to claim 1, wherein the pharmaceutical composition is stable for at least 6 months at 2-8° C. and 25° C./60% RH.

20. The article of manufacture according to claim 1, wherein the pharmaceutical composition has less than 1% total impurities after storage for 6 months at 2-8° C.

21. Method of administering a semaglutide composition for treating semaglutide sensitive diseases or disorders in a patient in need thereof, the method comprising: providing the article of manufacture according to claim 1, wherein the device comprises a concentration more than 27.5 mg/mL and less than 100 mg/mL semaglutide, and subcutaneously administering to the patient, a therapeutically effective dose of semaglutide from the article of manufacture at intervals of about once per week.

22. The method of administration semaglutide composition according claim 21, for treating semaglutide sensitive diseases or disorders selected from type 2 diabetes mellitus or obesity or to reduce the risk of major adverse cardiovascular events or noncirrhotic metabolic dysfunction-associated steatohepatitis (MASH) or chronic kidney disease in a patient in need thereof.
